(19) United States
(12) Patent Application Publication
(10) Pub. No.: US 2011/0081649 A1
(43) Pub. Date: Apr. 7, 2011

(54) DIAGNOSIS, STAGING AND MONITORING OF INFLAMMATORY BOWEL DISEASE

(75) Inventors: Magnus Thoern, Uppsala (SE); Ola Winqvist, Uppsala (SE)

(73) Assignee: ISS Immune System Stimulation AB, Soederkoeping (SE)

(21) Appl. No.: 12/671,318

(22) PCT Filed: Jul. 29, 2008
(86) PCT No.: PCT/SE2008/000464

§ 371 Date: Apr. 29, 2010

(30) Foreign Application Priority Data
Aug. 2, 2007 (SE) 0701807-0

(51) Int. Cl. C12Q 1/68; G01N 33/567

(52) U.S. Cl. 435/6; 435/7.21

(57) ABSTRACT

A method of differentiating between active and inactive IBD in a gastrointestinal mucosa sample or a sample from a sentinel lymph node draining gastrointestinal mucosa comprises preparing a suspension of single cells from the sample, analyzing the suspension for expression of the inflammation activation marker CD69 on CD4+ T helper cells using directly labelled fluorescent DC69 antibody; comparing the number of T helper cells expressing DC69 in the sample with that obtained from a corresponding sample of a healthy person, a significantly increased level of T helper cells expressing CDD69 signifying the presence of active IBD and a less than significantly increased level of T helper cells signifying the presence of inactive IBD. Also disclosed are methods of differentiating between ulcerative colitis (UC) and Crohn's disease (CD), of detecting UC and CD, and of determining the susceptibility of an IBD patient to steroid treatment.

### FIELD OF THE INVENTION

The present invention relates to diagnosis, staging, and monitoring of inflammatory bowel disease.

### BACKGROUND OF THE INVENTION

Inflammatory bowel disease (IBD) comprises Crohn's disease (CD) and ulcerative colitis (UC), both cause long-term damage to the intestine. Recently, microscopic colitis, collagenous colitis and lymphocytic colitis have been included in IBD. The combination of external antigens, impaired immune reactions and hereditary factors seem to contribute to the etiology of IBD, but the mechanisms initiating the intestinal inflammation remain unclear (1). So far, many potential antigens have been proposed, for example antigens derived through intestinal CMV infection (2) and persistent measles virus infection (3).

Most likely, mesenteric lymph nodes, draining the intestinal content, are crucial loci for the initiation and maintenance of inflammation in IBD. Antigens triggering inflammation are actively transported from the intestinal wall to the lymph nodes for presentation to the immune system (1, 4). Techniques have been developed to identify the first lymph nodes, which are draining pathological areas of the intestine and to harvest these nodes during surgery (5). The invention discloses assays constructed to delineate the different IBD diagnoses and stages based on cell surface marker expression and mRNA expression array profiling. These patterns are related to the type and activity of intestinal inflammation, and therefore are useful for diagnosis, staging and monitoring of disease activity, including response to therapy.

In Sweden, 1400 new persons are diagnosed with IBD every year. The number of newly diagnosed persons has been increasing in recent years. The IBD peak incidence occurs at 20 years of age (6,7). The intestine is an intricate microenvironment where immunocompetent cells meet with a broad variety of endogenous and exogenous factors. These interactions nurture a continuous, low grade inflammation reflected by the abundance of T cells in the lamina propria (8). T cells are constantly ready to act against harmful pathogens but they ignore innocuous dietary antigens or members of the normal resident bacterial flora. When the balance between tolerance and response in the intestinal immune system is abrogated, the consequence may be IBD.

CD and UC are the two major forms of IBD. In CD, any part of the bowel can be affected, but most common is affection of the ileocaecal region (50%) and colon (30%). About one third of the patients also have concomitant perianal disease. Several short segments of the small bowel may be affected, often known as skip lesions. Histopathologically, the inflammation is transmural; dense infiltrations of lymphocytes and macrophages with granulomas are seen in up to 60% of the patients. UC usually involves the rectum, and extends in a proximal direction, the extension varying between individual patients; the disease however always remains restricted to the colon. The term total colitis is used when the inflammation is also involving the right colonic flexure. Microscopically, mucosal inflammation is superficial and rich of lymphocytes and granulocytes. Ulcerations and crypt abscesses are common (1). Along with the symptoms from the prolonged inflammation such as diarrhoea (often containing blood), malnutrition, fever and pain, the patient is also at long-term risk of developing epithelial dysplasia and finally cancer in the intestine (9). About 15% of the IBD patients have indeterminate colitis and therefore a definite diagnosis of CD or UC cannot be set. Even more important, among patients with fulminant colitis, there is often uncertainty regarding diagnosis. A correct diagnosis is important when deciding about immediate medical treatment and, in a longer time perspective, about the best surgical treatment. Surgical treatments in CD include resection of inflamed segments, treatment of stenosis by stricturoplasties, colonic resection of affected segments or total colectomy and ileostomy in cases with severe colitis. Reconstructive surgery in CD is often an ileorectal anastomosis to restore intestinal continuity. A pelvic pouch is seldom chosen due to high risk of complications related to fistula formation in the ileoanal anastomotic area. Some patients end up with a permanent ileostomy. Patients with severe colitis due to UC are often treated by colectomy and ileostomy as an emergency procedure. About six months later either an ileorectal anastomosis or pelvic pouch can be chosen as definite surgical treatment but in some cases a permanent ileostomy may be the only possible solution.

Several lines of evidence imply that CD is a consequence of a disruption of tolerance towards the normal enteric flora. In humans, it has been shown that mononuclear cells in the lamina propria of patients respond to autologous faecal extracts, which is not the case in normal controls (10). The dependency on antigen for the T cell stimulation is apparent from observations of in vitro proliferation of lymphocytes from CD patients against sonicates of autologous intestinal bacteria, where the proliferation was inhibited by anti-MHC class II antibodies (11). Regarding UC, the role of the microenvironment is less clear, and the inflammation seems to be based on an autoimmune-like pathogenesis considering the dependency on immunoglobulins (IgG), the association with autoimmune disorders such as sclerosing cholangitis, the frequency of autoantibodies and the colon mucosal specificity (12). Hypothetically, an immune response towards the bowel may be an effect of insufficient inhibitory action of regulatory T cells (1, 4, 10).

Active UC is characterized by infiltration of activated granulocytes and monocytes/macrophages within the colonic mucosa. These infiltrating cells are major sources of inflammation-promoting cytokines. IBD patients may develop resistance towards medical treatment. Possible causes are changes in distribution of granulocytes, cytokines or functional characteristics of immune cells including the expression of steroid-receptors.

Today, the diagnosis and staging of IBD are based on a combination of clinical parameters, (albumin, SR, CRP, fever, number of stools) together with endoscopic criteria and histopathology. The inflammatory activity is evaluated by microscopy but only the general types and numbers of cells present in the biopsy are taken under consideration. Activation characteristics are not determined.

### OBJECTS OF THE INVENTION

It is an object of the invention to provide a method capable of differentiating between active and inactive Intestinal Bowel Disease (IBD).

It is another object of the invention to provide a method of diagnosis capable of differentiating between Crohn's disease (CD) and ulcerative colitis (UC) in an IBD patient.

It is further object of the invention to provide a method capable of differentiating between Crohn's disease and ulcerative colitis in a patient with indeterminate colitis.

Additional objects of the invention will become evident from the study of a summary of the invention, of preferred embodiments thereof illustrated in a drawing and the appended claims.

### SUMMARY OF THE INVENTION

The invention is based on the insight that intestinal antigens are continuously transported to the locoregional mesenteric lymph nodes by mucosal dendritic cells. In this process regulatory T cells can play a pivotal role in modulating T effector cells, thus preventing response to normal antigens.

In animal models two distinct pathways for T effector cell response can be distinguished. The first type of immune initiation is driven by activated T helper cells type 1 (Th1), which are characterized by the production of cytokines such as IL-12, IFN-gamma and TNF-alpha. The inflammatory site is dominated by cell-mediated infiltration of macrophages and cytotoxic T effector cells (CD8+). This type of inflammation resembles the lesions in CD. In the T helper cells type 2 (Th 2) immune activation, the humoral immune defense system (immunoglobulins) plays an important role in conjunction with cytotoxic T cells and cytokines such as IL-4 and IL-5 as well as IgG1 that are seen in the inflammatory sites. In animal models, Th 2 type inflammation shares features with human UC.

According to the present invention is disclosed a method of differentiating between Crohn's disease and ulcerative colitis in an IBD patient by submitting single cells isolated from biopsy samples of inflamed tissue to multicoloured flow cytometry. The determination of activation markers and cytokine production allows the status of the local immune response to be determined with high precision. The method of the invention is also useful for staging and for the evaluation of therapy in IBD-patients.

According to a first preferred aspect of the invention is provided a method of determining the presence of IBD in a person, the method comprising providing a biopsy sample from distal ileal or colonic mucosa suspected to be diseased or from a sentinel lymph node draining said distal ileal or colonic mucosa, determining the number of CD4 T cells expressing the CD 69 activation marker, and comparing said number of CD4 T cells with the number of corresponding T cells obtained from healthy persons and from persons having IBD in an active or inactive state.

According to a second preferred aspect of the invention is provided a method of differentiating between ulcerative colitis and Crohn's disease in an IBD patient. The method comprises providing a biopsy sample from diseased intestinal mucosa or a sentinel lymph node draining such mucosa, determining one or more of cytokines produced in immune activation driven by activated T helper cells type 1 (Th1) indicative of CD and one or more of cytokines produced in immune activation driven by activated T helper cells type 2 (Th2) indicative of UC, and comparing the amount of Th1 and Th2 cytokines with the amounts usually found in patients having manifest CD and/or UC. Preferred Th1 cytokines comprise IL-12, IFN-γ and TNF-α, in particular IFN-γ. Preferred Th2 cytokines comprise IL-4 and IL-5, in particular IL-4. In this method it is also preferred to independently or additionally determine IgG1 as a marker for Crohn's disease.

According to a preferred aspect of the invention mRNA expression profiling (13) of immune cells from IBD biopsies is used for diagnosis, staging, therapy and monitoring of therapy in IBD.

According to a further preferred aspect of the invention is disclosed a method of determining the susceptibility of an IBD patient to steroid treatment, comprising determination of glucocorticoid receptor expression in CD4+ T helper cells obtained from said patient.

In this application “staging” relates to the determination of the activation stage, severity of inflammation, type of inflammation, etc., in IBD, whereas “monitoring” relates to monitoring the development of IBD in a patient.

Further preferred aspects of the invention are disclosed in the following description of preferred embodiments thereof illustrated in a drawing comprising a number of figures, and in the appended claims.

### SHORT DESCRIPTION OF THE FIGURES

The invention will now be explained in more detail by reference to a number of preferred embodiments illustrated in a drawing, in which

FIG. 1 is a graph showing the investigation of single cell suspensions of biopsies from patients with active or inactive UC and from healthy controls using flow cytometry and antibodies against CD4 and the activation marker CD69, the mean fluorescent intensity on CD4+ cells being set off on the y-axis;

FIG. 2 is a graph correlating the percentage of activated CD69+CD4+ T cells seen in flow cytometry (FACS) investigated biopsies from healthy controls, patients with active and inactive UC and patients with inactive CD;

FIG. 3 is a graph correlating the IBD score of patients with different stages of disease with the percentage of activated CD69+CD4+ T cells in FACS investigated biopsies;

FIG. 4 is a graph showing the production of IFN-γ in cultured supernatants from non-sentinel node, sentinel node 1 and sentinel node 2 draining the inflammatory area of a CD patient;

FIG. 5 is a graph showing the production of IL-4 in cultured supernatants from non-sentinel nodes (NSLN) and sentinel nodes (SLN) draining the inflammatory area of a UC patient;

FIG. 6 is a graph showing decreased expression of intracellular levels of glucocorticoid receptors in CD4+ T helper cells from patients with IBD not responding to glucocorticoid treatment;

FIG. 7 is a graph showing increased Tbet expression in CD4+ T helper cells from the intestinal tract of a patient with active CD, whereas Tbet expression in an inactive phase is low;

FIG. 8 is a graph showing increased Tbet expression in CD4+ T helper cells from the intestinal tract from a patient with active CD, whereas Tbet expression in an inactive phase is low. Furthermore, Tbet expression in CD4+ T helper cells from a patient with UC is low compared to the Tbet expression seen in active CD;

FIG. 9 is a graph showing increased GATA-3 expression in CD4+ T helper cells from the intestinal tract from a patient with active UC, whereas GATA-3 expression in an inactive phase is low.

### DETAILED DESCRIPTION OF THE INVENTION

### Materials and Methods

Single cell suspensions were made from fresh biopsies. Flow cytometry (FACS) was performed using directly labelled fluorescent antibodies CD4, CD8, CD69, CD25, CD14, CD9, CD66b, CD19 using FITC, PE, PerCp, APC conjugates in combinations of four. Isotype antibodies where used as controls. In CD and UC sentinel nodes draining inflamed bowel and unaffected normal bowel were identified using a tracer substance, Patent Blue, injected around the area of inflammation for comparative analyses. A venous blood sample was also collected from each patient.

Analyses of cytokines IL-4 and IFN-γ were performed on single cell cultured supernatants from biopsies or sentinel nodes using sandwich ELISA (R&D systems).

Analyses of expression of glucocorticoid receptor were performed using a mouse anti human glucocorticoid receptor antibody followed by an anti-mouse IgG FITC conjugated detection antibody of saponin permeabilized single cells.

RNA isolation from single cell suspensions was performed using TRIZOL reagent (Invitrogen, Cat. No. 15596-026). 100 ng RNA per sample was included in a reverse transcriptase reaction using iScript™ cDNA Synthesis Kit according to manufacturer's protocol. Quantitative PCR was performed on an iCyclerIQ using 2X IQ™ SYBR® Green Supermix. Cycle thresholds were obtained using iCycler IQ™ Optical System Software Version 3.1 from BIO-RAD. Expression levels were normalized to RPII using the 2^{-ΔΔCt} method. Alternatively isolated RNA was subjected to cDNA synthesis, FAM labelling and thereafter to analysed using an Affymetrix human array chip.

### Primer Sequences QT-PCR:

| | |
|---|---|
| IFNγ QT Fwrd | GCAGGTCATTCAGATGTAGCGG |
| IFNγ QT Rev | TGTCTTCCTTGATGGTCTCCACAC |
| IL-4 QT Fwrd | CACAACTGAGAAGGGAAACCTTCTG |
| IL-4 QT Rev | CTCTCTCATGATCGTCTTTAGCCTTTC |
| T-bet QT Fwrd | CACTACAGGATGTTTGTGGACGTG |
| T-bet QT Rev | CCCCTTGTTGTTTGTGAGCTTTAG |
| GATA3 QT Fwrd | AACTGTCAGACCACCACAACCACAC |
| GATA3 QT Rev | GGATGCCTTCCTTCTTCATAGTCAGG |

### Example 1

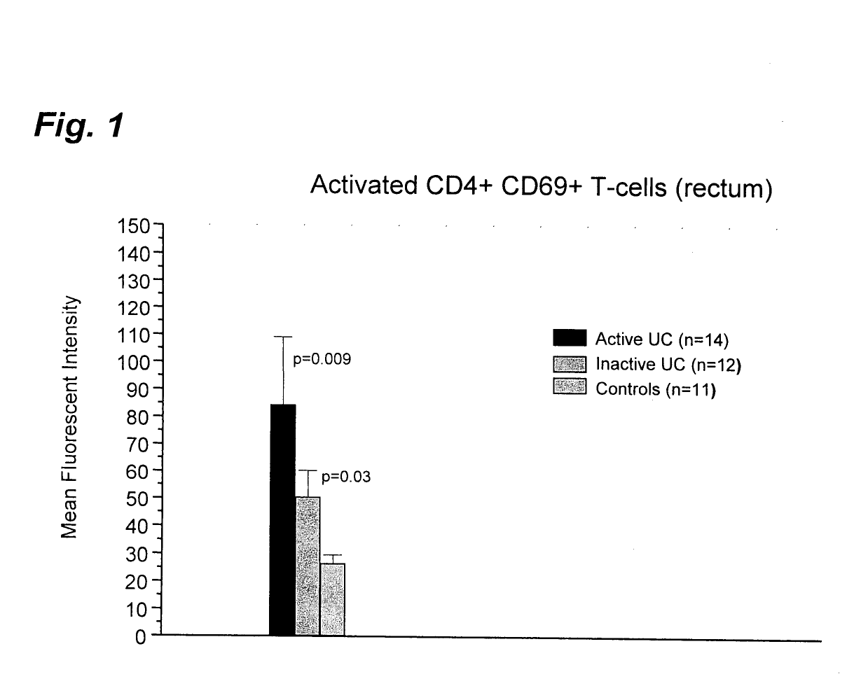

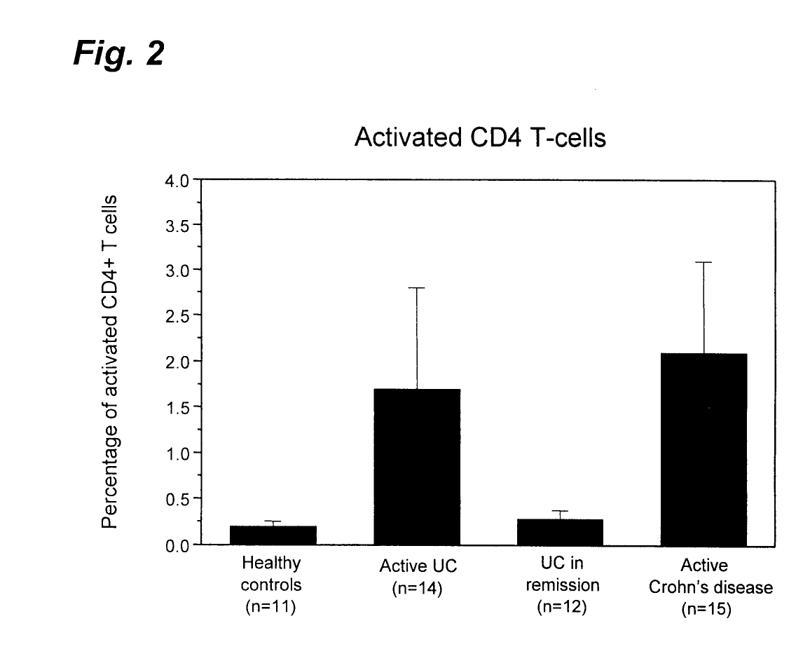

Investigation of immune cells in the mucosa or in the sentinel lymph nodes draining an area affected by IBD. Investigation by flow cytometry of immune cells in the mucosa or in the sentinel lymph nodes draining an area affected by IBD demonstrates that, in comparison with healthy controls, there is a significantly increased (p<0.009) expression of the activation marker CD69 on CD4+ T helper cells (FIG. 1) and of CD8+ cytotoxic T cells in patients with active IBD. Patients with inactive IBD (in the judgment of a competent pathologist) nevertheless display a significantly increased activation (p<0.03) of CD4+ T cells (FIG. 1) in addition to the increased expression of the activation marker CD69. The number of CD4+ CD69+ T cells is also increased in patients with active IBD (FIG. 2) both in ulcerative colitis and Crohn's disease in comparison with healthy controls and patients with reported inactive IBD.

### Example 2

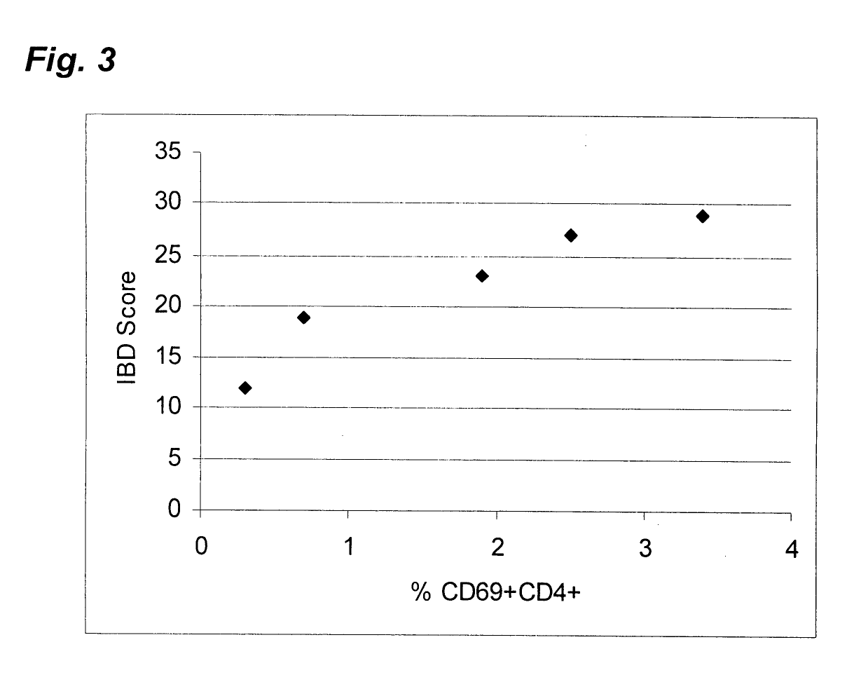

Correlation of the patients' IBD score with the flow cytometry profile. The patient's IBD score comprising S-albumin, CRP and the number of stools containing blood/day with the flow cytometry profile is shown in FIG. 3. The number of CD4+ T cells expressing the CD69 activation marker correlated well with the IBD score (SEO index).

SEO-index was utilized to correlate the results with disease activity. This index uses clinical (stool frequency and hematochezia) and laboratory parameters (Hb, albumin and erythrocyte sedimentation rate). In our material erythrocyte sedimentation rate was replaced by CRP (C-reactive protein).

### Example 3

Immune response in the sentinel nodes draining lesional areas of the intestine and in nodes draining unaffected segments in both CD and UC. Currently, the immune reaction in CD is regarded as a Th1 response, while UC may resemble an atypical Th2 activation. This view seems to be mainly based on animal models simulating colitis by artificial means. In humans, the characterization of the inflammatory pathways in IBD is limited; perfusion studies from the colon as well as the analysis of peripheral blood cells support some of the observations from animal models, but the general picture of the pathophysiology is incomplete. The determination of the immune response and cytokine patterns in the sentinel nodes draining lesional areas of the intestine and in nodes draining unaffected segments in both CD and UC according to the invention suggests that the release of cytokines can be used in CD and UC diagnosis.

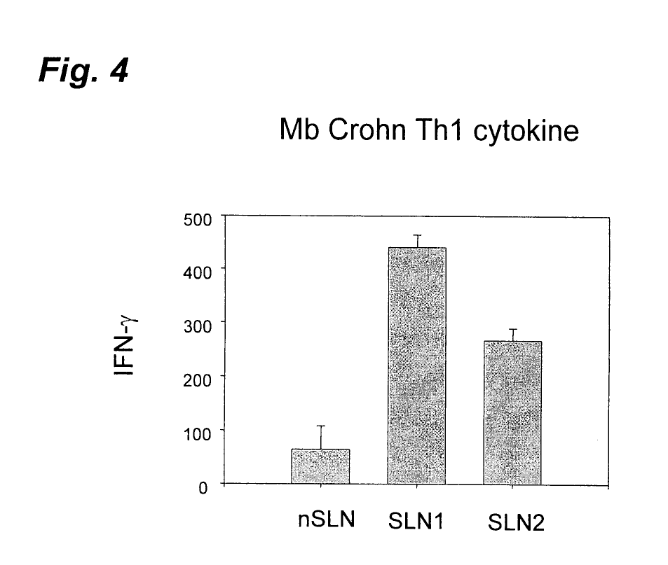

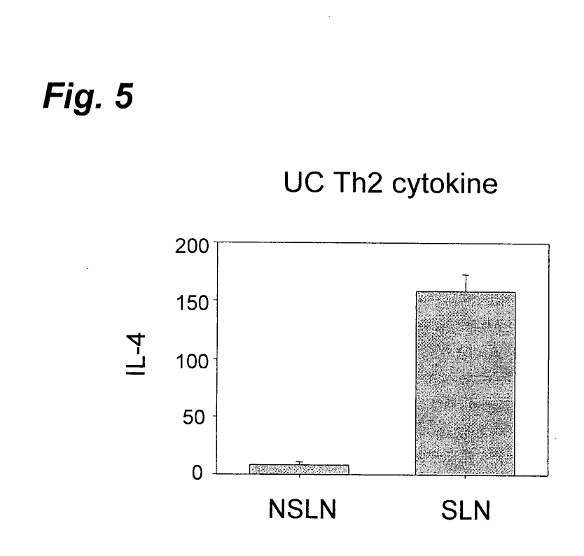

By prevailing standards 15% of IBD patients are considered to have indeterminate colitis, i.e. a definite diagnosis cannot be set. A correct diagnosis is however nevertheless important in deciding upon further treatment. Also, it is often impossible to differ between CD and UC in fulminant colitis, which may delay adequate medical treatment, and necessitate early surgical intervention with colectomy. High amounts of IFN-γ have now been identified in gastrointestinal inflammatory sites of patients with CD (FIG. 4). In contrast, gastrointestinal inflammatory sites of patients with UC have been found to contain high amounts of the Th2 cytokine IL-4 (FIG. 5). These findings allow to correlate a high IFN-γ content in a gastrointestinal biopsy sample with CD, and the production of high amounts of IL-4 with UC. These markers allow to differentiate between CD and UC also in fulminant colitis where a correct diagnosis often is difficult. Information obtained from intestinal biopsies may be used to provide a correct diagnosis early, in order to tailor medical treatment more precisely and to prevent surgical intervention with total colectomy and ileostomy in some patients.

In a similar manner multicoloured flow cytometry analysis of activation markers of other immune cells such as neutrophils, eosinophils, NK cells, NKT cells, T regulatory cells, and B cells can be useful in the diagnosis, staging and monitoring of IBD.

### Example 4

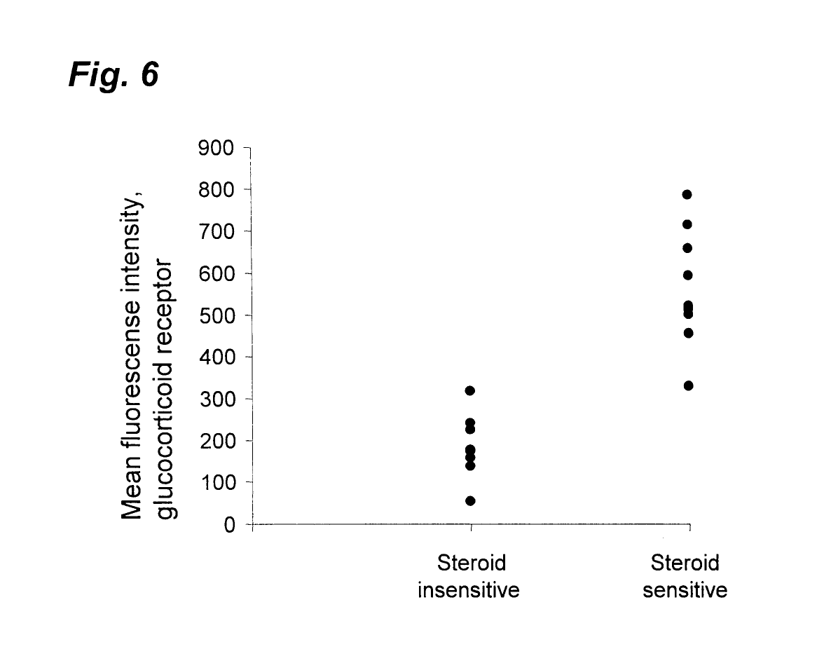

Determination of steroid receptor expression profile. It is a well-known problem that some patients with IBD may become resistant to steroid treatment. According to the present invention is disclosed an investigation of steroid receptor-expression profile in IBD-patients using Western blot or intracellular flow cytometry of single cells from biopsies to determine whether these cells are sensitive to steroid treatment. Intestinal biopsies or single cell suspensions from patients with steroid sensitive responses or patients not sensitive to steroid treatment were investigated by intracellular flow cytometry for the degree of steroid receptor expression. All patients with steroid refractory IBD expressed lower levels of glucocorticoid receptor, whereas patients who responded with intestinal healing and lower SEO index upon steroid treatment expressed higher levels (FIG. 6). Thus, expression levels of steroid receptors in intestinal lymphocytes correlate with susceptibility to treatment.

### Example 5

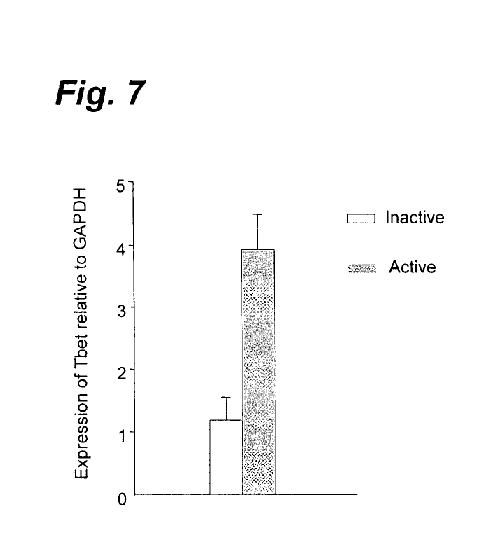

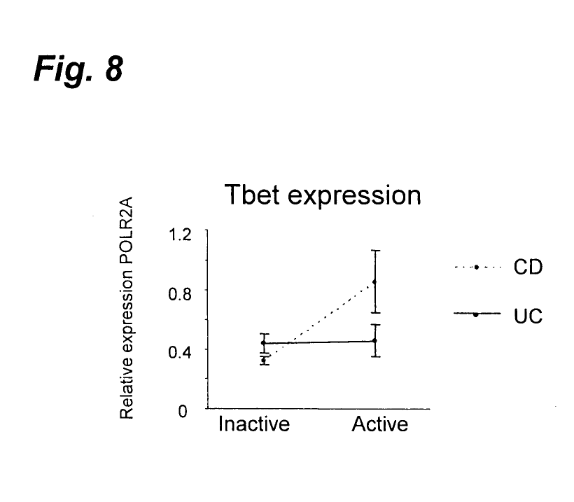

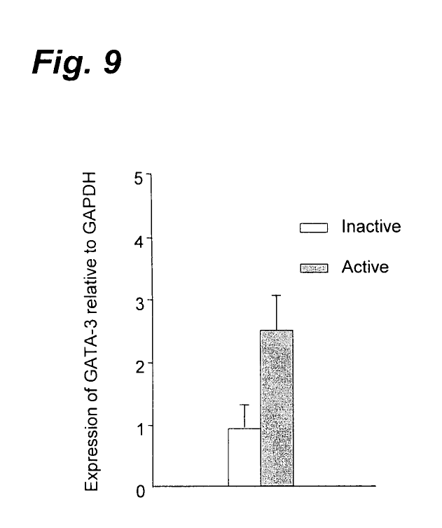

Cytokine expression array profiling. According to the invention a more detailed picture of the cytokine expression pattern of immune cells in biopsies from IBD patients is obtained by expression array profiling. This kind of profiling was performed using a 20 k Affymetrix chip. The Th1 activation pattern found in CD patients seems to be caused by up-regulation of the mRNAs for CD69 and Tbet, IFN-γ and TIM-3. On the contrary, a Th2 expression pattern is seen in UC patients where, in addition to CD69, the up-regulation of GATA-3 and IL-4, IL-5 was noted. The array findings were verified by real time quantitative PCR (QT-PCR). Accordingly, cells from CD patients displayed a higher expression level of the Tbet transcript (FIG. 8), whereas the GATA-3 transcript was elevated in cells from patients with UC (FIG. 9). The expression of Tbet was furthermore found to be substantially increased in patients with CD in an active phase (FIG. 7). Therefore, based on a panel of expression profiles, Th1 dominance in CD can be predicted by increased expression of Tbet, IFN-y and TIM-3. Consequently, Th2 dominance and UC can be predicted by the increased expression of the GATA-3 and IL-4, IL-5 transcripts.

### REFERENCES

1. Bouma G, Strober W. The immunological and genetic basis of inflammatory bowel disease. Nature Reviews Immunology 2003; 3: 521-533.
2. Rahbar A, Boström L, Lagerstedt U, Magnusson I, Söderberg-Naucler C, Sundqvist V A. Evidence of active cytomegalovirus infection and increased production of IL-6 in tissue specimens obtained from patients with inflammatory bowel diseases. Inflamm Bowel Dis 2003; 3:154-61.
3. Wakefield E, A J, Ekblom A, Dhillon A P, Pittilo R M, Pounder R E. Crohn's disease: pathogenesis and persistent measles virus infection. Gastroenterology 108:911-916, 1995.
4. Toms C, Powrie F. Control of intestinal inflammation by regulatory T cells. Microbes and infection. 2001; 3: 929-935.
5. Thorn M. Lymphatic mapping and sentinel node biopsy: is the method applicable to patients with colorectal and gastric cancer? Eur J Surg. 2000; 166: 755-758.
6. Lapidus A, Bernell O, Hellers G, Persson P G, Löfberg R. Incidence of Crohn's disease in Stockholm County 1955-1989. Gut. 1997; 41: 480-486.
7. Tysk C, Jarnerot G. Ulcerative proctocolitis in Örebro, Sweden. A retrospective epidemiologic study, 1963-1987. Scand J Gastroenterol. 1992; 27: 945-50.
8. Wittig B M, Zeitz M. The gut as an organ of immunology. International Journal of Colorectal Disease. 2002: e-journal.
9. Ekbom A, Helmick C, Zack M, Adami H O. Ulcerative colitis and colorectal cancer. A population-based study. N Engl J Med 1990; 323: 1228-33.
10. Strober W, Kelsall, B. To be responsive or not to be responsive, that is the mucosal question. Gastroenterology 1998; 114: 214-217.
11. Duchmann R, Kaiser I, Hermann E, Mayet W, Ewe K, Meyer-zum-Buschenfelde K H. Tolerance exists towards resident intestinal flora but is broken in active inflammatory bowel disease. Clin Exp Immunol 1995; 102: 448-455.
12. Macdonald T T, Monteleone G, Pender S L F. Recent developments in the immunology of inflammatory bowel disease. Scan J. Immunol. 2000; 51: 2-9.
13. DeRisi J, Penland L, Brown P O, Bittner M L, Meltzer P S, Ray M, Chen Y, Su Y A, Trent J M. Use of a cDNA microarray to analyse gene expression patterns in human cancer. Nat Genet. 1996 14(4):457-60.

1. A method of differentiating between active and inactive inflammatory bowel disease (IBD) in a patient, comprising providing a biopsy sample of gastrointestinal mucosa suspected to be diseased or from a sentinel lymph node draining said gastrointestinal mucosa; preparing a suspension of single cells from the sample; analyzing the suspension of single cells for expression of the inflammation activation marker CD69 on CD4+ T helper cells using directly labelled fluorescent CD69 antibody; comparing the number of T helper cells expressing CD69 so determined with the number of T helper cells expressing CD69 determined in a corresponding suspension of single cells obtained from intestinal tissue of a healthy person; wherein a significantly increased level of T helper cells expressing CD69 signifies the presence of active IBD, whereas a less significantly increased level of T signifies the presence of inactive IBD.

2. The method of claim 1, wherein analysis is by flow cytometry.

3. (canceled)

4. The method claim 1, wherein the antibody is a fluorescent antibody.

5. The method of claim 1, wherein said significantly increased level is characterized by p<0.009.

6. The method of claim 1, wherein said less significantly increased level is characterized by 0.03<p>0.009.

7. A method of differentiating between ulcerative colitis and Crohn's disease in an IBD patient, comprising providing a patient biopsy sample obtained from diseased intestinal mucosa or a sentinel lymph node draining such mucosa, preparing a suspension of single cells from the sample; determining in the suspension or a supernatant thereof one or more of cytokines produced in immune activation driven by activated T helper cells type 1 (ThI) indicative of Crohn's disease and one or more of cytokines produced in immune activation driven by activated T helper cells type 2 (Th2) indicative of ulcerative colitis, comparing the determined amounts of ThI and Th2 cytokines with corresponding amounts found in patients having manifest Crohn's disease and/or ulcerative colitis.

8. The method of claim 7, wherein a cytokine produced in immune activation driven by activated T helper cells type 1 is selected from IL-12, IFN-γ and TNF-α.

9. The method of claim 7, wherein a cytokine produced in immune activation driven by activated T helper cells type 2 is selected from IL-4 and IL-5.

10. The method of claim 7, additionally comprising determining IgG1 in the sample as a marker for Crohn's disease.

11. The method of claim 8, wherein any of IL-12, IFN-γ and TNF-α is determined in said supernatant.

12. The method of claim 9, wherein any of IL-4 and IL-5 is determined in said supernatant.

13. A method of determining the presence of Crohn's disease in a patient, comprising providing a biopsy sample of gastrointestinal mucosa suspected to be diseased or from a sentinel lymph node draining said gastrointestinal mucosa; preparing a suspension of single cells from the sample; isolating mRNA from the cell suspension; analyzing the isolated mRNA for mRNA of two or more of CD69, Tbet, IFN-γ, TIM-3; comparing the result with that of the analysis of mRNA of corresponding two or more of CD69, Tbet, IFN-γ, TIM-3 in correspondingly obtained mRNA from a healthy person, up-regulation of two or more of mRNA of CD69, Tbet, IFN-γ, TIM-3 in the patient mRNA sample being indicative of Crohn's disease.

14. (canceled)

15. A method of determining the presence of ulcerative colitis in a patient, comprising providing a biopsy sample of gastrointestinal mucosa suspected to be diseased or from a sentinel lymph node draining said gastrointestinal mucosa; preparing a suspension of single cells from the sample; isolating mRNA from the cell suspension; analyzing the isolated mRNA for mRNA of two or more of CD69, GATA-3, IL-4, IL-5; comparing the result with that of the analysis of mRNA of corresponding two or more of CD69, GATA-3, IL-4, IL-5 in correspondingly obtained mRNA from a healthy person, up-regulation of two or more of mRNA of CD69, GATA-3, IL-4, IL-5 in the patient mRNA sample being indicative of ulcerative colitis.

16-19. (canceled)

20. A method of determining the susceptibility of an IBD patient to steroid treatment, comprising determination of glucocorticoid receptor expression in CD4+ T helper cells obtained from said patient.

21. The method of claim 20, wherein said determined expression is compared with a corresponding expression in an IBD patient or a group of IBD patients responding to steroid treatment and/or in an IBD patient or a group of IBD patients not responding to steroid treatment.

22. The method of claim 21, wherein a 50 percent increase of said determined expression in said IBD patient in respect of the average expression in a group of non-responding IBD patients is indicative by 80 percent or more of said patient being susceptible to steroid treatment.

23. The method of claim 22, wherein the only criteria for selecting said group of non-responding IBD patients is their unresponsiveness to steroid treatment.


SEQUENCE LISTING (8 sequences)

SEQ ID NO: 1 - DNA, 22 bp, artificial
  Primer
gcaggtcattcagatgtagcgg

SEQ ID NO: 2 - DNA, 24 bp, artificial
  Primer
tgtcttccttgatggtctccacac

SEQ ID NO: 3 - DNA, 25 bp, Artificial
  Primer
cacaactgagaagggaaaccttctg

SEQ ID NO: 4 - DNA, 27 bp, Artificial
  Primer
ctctctcatgatcgtctttagcctttc

SEQ ID NO: 5 - DNA, 24 bp, Artificial
  Primer
cactacaggatgtttgtggacgtg

SEQ ID NO: 6 - DNA, 24 bp, Artificial
  Primer
ccccttgttgtttgtgagctttag

SEQ ID NO: 7 - DNA, 25 bp, Artificial
  Primer
aactgtcagaccaccacaaccacac

SEQ ID NO: 8 - DNA, 26 bp, Artificial
  Primer
ggatgccttccttcttcatagtcagg